(19) United States
(12) Patent Application Publication
(10) Pub. No.: US 2010/0009395 A1
(43) Pub. Date: Jan. 14, 2010

(54) LUMINESCENCE ASSAY UTILIZING A GENETICALLY MODIFIED CELL LINE

(76) Inventors: Rama P. Ramanujam, Columbus, OH (US); Shrikant Anant, Oklahoma City, OK (US)

Correspondence Address: QUARLES & BRADY LLP, 411 E. WISCONSIN AVENUE, SUITE 2040, MILWAUKEE, WI, 53202-4497, US

(21) Appl. No.: 12/374,824

(22) PCT Filed: Jul. 24, 2007
(86) PCT No.: PCT/US07/16615

§ 371 Date: Jan. 23, 2009

Related U.S. Application Data
(60) Provisional application No. 60/833,545, filed on Jul. 26, 2006.

(51) Int. Cl. C12Q 1/66; C12Q 1/02

(52) U.S. Cl. 435/8; 435/29

(57) ABSTRACT

An assay method to identify agents that will reduce the inflammation associated with many diseases, providing a method to determine compliance of patients to clinical protocols. The fundamental tool of the inventive method is luminescence. Genetically modified cells are used to express a complex revealing the potential of certain compounds to prevent or reduce adverse effects. More specifically the invention is a method for the determination of inhibition of a chemical compound comprising: culturing genetically modified cells which express an indicator-luminescent complex; placing said complex in the presence of an agent that essentially totally degrades said complex; measuring the luminescence of the resulting reaction; collecting a sample from a mammal consuming a complementary and alternative medicine (CAM); placing said sample in the presence of said complex; and comparing the level of luminescence from step (c) with the luminescence from step (e) to determine the inhibition of said chemical compound.

### RELATED APPLICATION AND CLAIM TO PRIORITY

This application claims priority to U.S. Provisional Patent Application No. 60/833,545; filed Jul. 27, 2006.

### FIELD OF THE INVENTION

In general, this invention relates to an assay for the presence of naturally occurring chemical entities or metabolites thereof in body fluids through the use of bioluminescence imaging. More specifically, the present invention is directed to the use of a genetically modified cell line to express a desired compound bonded to a chemical entity that is luminescent. A reduction in the level of luminescence, compared to a standard, indicates the presence of the naturally occurring chemical entity.

### BACKGROUND OF THE INVENTION

Recently there has been much interest in the use of complementary and alternative medicines (hereinafter CAM) for the treatment and prevention of disease. Proving the efficacy of CAM therapies can be difficult as outpatient trials require the patients to adhere to the therapeutic protocols. Proof of adherence to the protocols presently includes patient reports, pill counts and sophisticated analytical analysis. These methodologies are time consuming and expensive. In contrast, the present invention provides inexpensive, non-invasive methods to detect CAM in body fluids such as urine, serum, saliva, sweat, semen and blood.

A study by the American Cancer Society researchers indicate that people that consume large amounts of red meat or processed meats are at a higher risk for colon cancer. Alternatively, there has been a prevailing belief from a number of studies that high intake of fruits and vegetables may reduce the risk of colon cancers. There is a rising trend for many of the common cancers including colorectal, breast and prostate cancer, which are linked to the “Western lifestyle;” a relatively sedentary way of life with diet low in fiber and fresh fruit and vegetables but rich in calories, meat, fat, salt, additives and alcohol. Certain food constituents and specific nutrients believed to protect against cancer are dietary fiber, phytochemicals, and vitamins A, C and E. In addition, evidence suggests that certain chemicals in plant foods sources may prevent cancer.

Epidemiological cancer studies and laboratory tests of animals have indicated that consumption of spices, fruits, vegetables and whole grains can reduce the risk of certain cancers and inflammation. Long term inflammation is now being recognized as a major cause of cancer. Furthermore, inflammation plays a major role in other diseases such as arthritis, autoimmune diseases, Alzheimer's disease, neurological diseases, pulmonary diseases, cardiovascular diseases and diabetes. Hence, using this technology, we may be able to test for phytochemicals that may prevent or cure these diseases. However, traditional dietary recommendations lack adherence monitoring systems to accurately measure the positive or negative outcomes. Accordingly, one aspect of this invention is to develop a rapid, real-time, gene-expression based luminescence-assay to measure the effectiveness of bioactive constituents of CAM agents against inflammatory mediators in cultured cells.

Representative CAM agents that can be detected using this invention include spices such as turmeric extracts, fruits such as black raspberry extracts and cruciferous vegetables such as broccoli extracts.

At the cell and molecular level, inflammation is accompanied by the secretion of pro-inflammatory cytokines, such as tumor necrosis factor-alpha, interleukin-1, IL-6, IL-12, and gamma-interferon. This increased production of cytokines and the subsequent elevation in reactive nitrogen and oxygen radicals are recognized hallmarks of inflammation.

The inflammatory process is also regulated by a negative feedback mechanism and closely followed by the secretion of anti-inflammatory cytokines to reduce the accumulation of reactive nitrogen and oxygen radicals. Inhibition of these pathways can be achieved by CAM therapies, which is why the National Center for Complementary and Alternative Medicines (NCCAM) and the National Institute of Health (NIH) are initiating clinical trials with CAM agents. Hence, we believe the technique developed in this invention will help in objectively assessing patient adherence to CAM studies.

The transcription factor NF-kappa B is a key regulator of normal cellular processes, such as immune and inflammatory responses, developmental processes, cellular growth, and apoptosis. This factor is also persistently active in a number of disease states, including cancer, arthritis, chronic inflammation, asthma, neurodegenerative diseases, and heart disease. The activity of NF-kappa B is tightly regulated by interaction with an inhibitory I-kappa B protein complex comprising I-kappa B beta, I-kappa B beta and I-kappa B gamma. Role of NF-kappa B in immune and inflammatory responses is well documented. A major contribution to the state of the art has been accomplished through the discovery that certain CAM agents inhibit the degradation of I-kappa B, and that this inhibition can be used in a luminescence assay to screen potential drug candidates and ensure patient compliance with study protocols. The present invention saves time and money over the currently used ‘Pill-Count’ or HPLC/MS analyses.

From drug discovery to determining efficacy and compliance to a clinical study protocol, there is a need for a highly sensitive and reproducible assay. Currently, there are assays to identify drugs and their metabolites in the body fluids. The technique that is most commonly used is high pressure liquid chromatography (HPLC) combined with mass spectrophotometry analyses. Unfortunately, these are both time consuming and labor intensive. In this invention, we have developed, in a preferred embodiment, an inexpensive and simple luciferase assay to demonstrate activity of CAM agents after ingestion.

### SUMMARY OF THE INVENTION

CAM agent research and development will benefit from inexpensive and non-invasive methods of analysis. Currently, urinalysis is the classic means to detect substance use, and has grown due to technical advances in such testing. However, none of the current techniques are high throughput and cost-effective for determining adherence to CAM protocols. In one embodiment of the invention, it has been discovered that the use of a functional high-throughput gene expression based urinalysis assay is able to detect low levels of CAM agents.

Thus, there is disclosed a method for the determination of inhibition of a chemical compound comprising: a) culturing genetically modified cells which express an indicator-luminescent complex; b) placing the complex in the presence of an agent that essentially totally degrades said complex; c) measuring the luminescence of the resulting reaction; d) collecting a sample from a mammal consuming a complementary and alternative medicine (CAM); e) placing said sample in the presence of said complex; f) measuring the luminance of the reaction product of step (e); and (g) comparing the level of luminescence from step (c) with the luminance from step (e) to determine the inhibition of said chemical compound.

More specifically, the complexes can be measured from the supernatant of said genetically modified cells. Further, the method of the present invention can use a method of determination selected from the group consisting of an luciferase assay, Cox-2 assay, a DNA binding assay, an enzyme-linked immuno-sorbant assay, an antibody-RNA blot assay and an infra-red quantum dot label assay.

In an embodiment of the present invention the cells are of mammalian origin selected from the group consisting of liver cells, kidney cells, brain cells, fibroblast cells, nerve cells, skin cells, lung cells, spleen cells, endometrial cells, cardiac cells, stomach cells, breast cells, stem cells and a hematopoietic cell; and cell lines derived from any of these cells or of cancer cells.

A major aspect of the invention resides in the determination of the reduction of the luminescence of the complex when placed in the presence of the body fluid of a mammal consuming the CAM agent compared to the luminescence obtained with an agent that essentially totally degrades the indicator-luminescent complex. The preferred body fluid is urine, however, serum, saliva and semen can also be used.

### DETAILED DESCRIPTION OF THE INVENTION

The present invention will be more clearly understood from the following description of certain preferred embodiments. The invention is broadly applicable to identifying compounds which are capable of entering into binding reactions in living cells to prevent or lessen the inflammatory cascade associated with TNF.

A critical cellular factor that is involved in controlling many normal cellular and organism processes, including immune and inflammatory responses, cellular growth, and apoptosis is the transcription factor NF-kappa B. Moreover, NF-kappa B is continually active in many diseases, including cancer, arthritis, chronic inflammation, asthma, neurodegenerative diseases and heart disease. Under physiological conditions, NF-kappa B is present as a latent, inactive, I-kappa B-bound complex in the cytoplasm. I-kappa B is a complex of three subunits. Following stimulation by an extra cellular signal, the alpha subunit of I-kappa B is targeted for phosphorylation followed by ubiquitination and proteosomal degradation. Phosphorylation of I-kappa B kappa releases it from the NF-kappa B complex and the unmasked NF-kappa B complex can then enter the nucleus. Preferably, CAM agents, such as curcumin, inhibit I-kappa B degradation, thereby inhibiting NF-kappa B activation and suppressing pathophysiological processes.

An aspect of this invention is to develop sensitive, non-invasive kits and assays for use in the detection of biologically active CAM agents. This invention will also allow a means of assessing adherence to clinical trial protocols. Towards this goal, this invention describes an in vitro assay system using I-kappa B stabilization to determine which CAM agents are effective in preventing or alleviating disease associated with the TNF cascade.

Representative CAM agents that have demonstrated a positive effect using the present invention include turmeric, black raspberries and broccoli. These and other CAM agents are potent anti-inflammatory agents. This is because they are effective in reducing I-kappa B degradation. I-kappa B is a critical factor involved in the inflammatory pathway and plays a vital role in NF-kappa B activity.

The following examples demonstrate that an I- kappa B kappa-luciferase (IkB-Luc) fusion protein is a novel surrogate marker for the determination of I-kappa B kappa degradation in cells. Further, the following examples demonstrate that certain CAM agents inhibit the tumor necrosis factor (TNF)-alpha-mediated degradation of IkB-Luc. As a control, it has been observed that samples from a subject not consuming a CAM agent, do not affect the IkB-Luc degradation activity. In contrast, samples collected subsequent to ingestion of a CAM agent, such as cucurmin, significantly inhibited the IkB-Luc degradation.

The concentration of the analyte of interest from the CAM agent consuming mammal will be present in the body fluid sample at concentrations typically above 0.01 parts per million. Analogs of the analyte of interest may also be used in the present invention, which can be natural or synthetic These are typically compounds which have binding properties comparable to the analyte, but can also be compounds of higher or lower binding capability.

Numerous methods and systems have been developed for the detection of analytes of interest in biochemical, biological, chemical and botanical substances. Methods and systems which are capable of measuring trace amounts of chemicals, drugs, metabolites, microorganisms, pharmaceuticals, hormones, viruses, antibodies, nucleic acids and other proteins are of great value to scientists, clinicians and regulators. In general, the existence of an analyte of interest is indicated by the presence or absence of an observable “label” attached to one or more of the binding materials. Of particular interest are labels which can be made to luminescence through chemical, physical, photochemical and electrochemical means.

### EXAMPLE 1

In this experiment turmeric extracts were investigated to determine if they would inhibit intestinal adenomas in APCmin/+mice. The results confirm that the extract possesses potent inhibitory effects on intestinal adenomas. The turmeric used was (Curcuma longa L of the family Zingiberaceae) that was isolated from a farm in South India that is grown under organic conditions.

As a control it was been determined that urine of a human volunteer before ingestion of curcumin had no significant effect in TNF-alpha-mediated degradation of the Ikb-Luc protein in HCT-116 cells. However, after two hours following ingestion of 8 g curcumin, urine samples, after analysis, determined that there was a significant increase in the protection of the IkB-Luc from TNF-alpha-mediated degradation. This supports that the present invention can be used to determine useful CAM agents and the determination that a subject is following the prescribed protocol.

### EXAMPLE 2

In this experiment urine and serum samples at baseline (control) without CAM treatment will be obtained at different times of the day and the effect of increasing concentration of this baseline urine and serum on IkB-Luc degradation activity will be determined. Second, the control mice urine will be spiked with increasing concentrations of curcumin or ellagic acid/anthocyanin (from Black Raspberry) or indole-3-carbinol or sulforaphane (from broccoli) and determination made of their effects in inhibiting I-kappaB degradation. Finally, the mice will be fed increasing amounts of turmeric, black raspberry or broccoli extracts in their chow diet. Urine samples will be collected over a period of time to determine the minimal quantity of the CAM agent required in the diet to obtain significant levels in the urine that inhibits IkB-Luc degradation.

Extracts were prepared from organically grown plants which are free of any synthetically compounded fertilizers, pesticides and growth regulators. The extracts were prepared by homogenizing the plant/rhizome with two volumes of distilled water, and the homogenate lyophilized to a fine powder. The lyophilized materials were powdered with a mortar and pestle and extracted with 80% ethanol and stirred overnight at 4° C. The liquid was filtered and rotary evaporated until a solid residue remained. The residue was suspended in ethanol and stored at −20° C.

The in-vitro assay used in this invention is preferably based on the I-kB luciferase (I-kB-Luc) plasmid construct. This construct has been transfected into HeLa and HCT-116 cells. These stably transfected cells are the basis of the present invention.

Typically, the cells were plated in a 24-well dish and allowed to grow for 24 hours; they were then treated with 10 ng/ml TNF-alpha. The substrate for luciferase, D-luciferin was also added to the cells at the same time, and the cells were incubated in extremely light-tight, low-background imaging chamber, at 37° C. Photon luminescence emitted from the cells is detected with a back-thinned CCD camera, designed for high-efficiency photon detection, particularly in the important red region of the spectrum.

The results show that the TNF-alpha rapidly induced degradation of I-kB-Luc, but not the control firefly luciferase (Fluc) that lacked I-kappa B, suggesting specificity of TNF-alpha in the process.

In this experiment it was also discovered that curcumin is an inhibitor of COX-2 gene expression. Endogenous levels of COX-2 were determined following addition of the three botanical extracts. After incubating the HCT-116 cells with the different fractions for 1 hour, the cells were treated with TNF-alpha, EGF or IL-1 kappa (all known inducers of COX-2 expression) for an additional 1 hour to induce high levels of COX-2 expression. The results demonstrate that a CAM agent, such as curcumin, can reduce expression of COX-2.

The results of this experiment demonstrate that the inventive in vitro imaging assay is useful to detect biologically active CAM agents, their constituents or metabolites in body fluids and to assess adherence to protocols involving these agents. These data sets demonstrate the feasibility of this approach to determine the activity of a compound that regulates NF-kappa B activity in cells. Thus, this invention can be used to produce a high throughput assay to determine the activity of the various extracts.

### EXAMPLE 3

This experiment is designed to validate a rapid screening assay to determine adherence of clinical subjects to a study protocol ingesting CAM agents. In this experiment, the subject gave a urine sample in the morning, followed by ingestion of 8 gms. of curcumin. Two hours following ingestion, a urine sample was collected. Samples after ingestion were incubated with the cells expressing IkB-Luc. The results indicated that curcumin reduced the TNF degradation of I kappa B-Luc. Similar experiments were also conducted with a black raspberry extract to demonstrate that a patient is within compliance with the CAM-related clinical study.

### EXAMPLE 4

In this experiment the degradation of I-kappa B was investigated. The I-kappa B was stably expressed as a fusion protein with the firefly luciferase in HCT-116 colon cancer cells. Presence of I-kappa B can then be monitored by luminescence activity. It was determined that the fusion protein is subject to similar levels of degradation in response to a stimulus such as tumor necrosis factor-alpha (TNF-alpha), a known inducer of phosphorylation and degradation of I-kappa B, as would be the case with the native of I-kappa B. Western blot analyses of total cell lysates from TNF-alpha-treated cells were subjected to western blot analyses. Both, native I-kappa B and I-kappa B-luciferase (IkB-Luc) levels were decreased within 20 minutes of incubation with TNF-alpha.

Further, similar experiments were conducted in HeLa cells, a cervical carcinoma cell line; and obtained similar results. These data demonstrate that the I-kB-Luc fusion protein responds to external stimuli in manner similar to that observed with endogenous I-kB. The imaging assay cells were plated in a 24 well dish and allowed to grow for 24 hours; they were then treated with 10 ng/ml TNF-alpha, the substrate for luciferase, D-luciferin was also added at the same time, the cells were then incubated in a light-tight, low background imaging chamber, at 37° C.

Photon luminescence emitted from the cells was detected with a back-thinned CCD camera, designed for high-efficiency photon detection. The CCD is cooled and the electronic readout is optimized so that the data gathered extremely low noise. TNF-alpha rapidly induced degradation of I-kappa B-Luc, but not the control firefly luciferase (Fluc) that lacked I-kappa B. The data colleted supports that the present invention can determine the activity of a compound that regulates NF-kappa B activity in cells.

### EXAMPLE 5

HCT-116 cells stably expressing I-kB-Luc were incubated for 2 hours with increasing concentrations (0-1000 μl) control or post-curcumin ingested urine. Subsequently, some of the cells were treated with TNF-alpha. The level of I-kB degradation was determined by the luciferase assay. The results indicate that the control urine does not have any effect on I-kB degradation. However, the post-curcumin urine significantly suppressed the TNF-alpha-mediated I-kB-luciferase degradation. This experiment demonstrates that the present invention can be used as a screening assay to determine adherence of the clinical subjects to a study protocol with CAM agents. Similar experiments were conducted with black raspberry extracts. The resulting data supports the use of the inventive method to confirm compliance to CAM agent treatment.

While the methods and materials herein have been described in terms of preferred embodiments, it will be apparent that variations may be applied to the methods and/or materials without departing from the concept, spirit and scope of the invention.

### INDUSTRIAL APPLICABILITY

The medical community is in constant need of new and efficacious means of preventing or reducing disease. The present invention discloses and claims a tool to identify and determine CAM agents that are useful to that purpose.

We claim:

1. A method for the determination of inhibition of a chemical compound comprising:
a) culturing genetically modified cells which express an indicator-luminescent complex;
b) placing said complex in the presence of an agent that essentially totally degrades said complex;
c) measuring the luminescence of the resulting reaction;
d) collecting a sample from a mammal consuming a complementary and alternative medicine (CAM);
e) placing said sample in the presence of said complex and measuring the luminescence of the resulting reaction; and
f) comparing the level of luminescence from step (c) with the luminescence from step (e) to determine the inhibition of said chemical compound.

2. The method according to claim 1 wherein said chemical compound is selected from the group consisting of complementary and alternative medicine agents (CAM agent), and anti-inflammatory agents.

3. The method according claim 1 wherein said genetically modified cells are selected from the group consisting of liver, brain fibroblast, nerve, skin, lung, spleen, endometrial, cardiac, stomach, breast and stem cells.

4. The method according to claim 1 wherein said complex is isolated from the supernatant of said cell culture.

5. The method according to claim 1 wherein said luminescence is bio-luminescence.

6. The method according to claim 1 wherein said complex comprises a firefly luciferase fragment.

7. The method according to claim 1 wherein said sample is selected from the group comprising urine, serum, saliva and semen.

8. The method according to claim 1 wherein said complex is at least one protein fused to a luciferase fragment.

9. The method according to claim 1 wherein said genetically modified cells comprises cancer cells.
